(19) United States
(12) Patent Application Publication
(10) Pub. No.: US 2023/0050513 A1
(43) Pub. Date: Feb. 16, 2023

(54) SCREENING SYSTEM AND METHOD FOR ACQUIRING AND PROCESSING GENOMIC INFORMATION FOR GENERATING GENE VARIANT INTERPRETATIONS

(71) Applicant: CONGENICA LTD., Cambridge (GB)

(72) Inventors: Sandro Morganella, Upper Cambourne (GB); Yacine Dahman, Cambridge (GB); Laura Ponting, Cambridge (GB); Emily Mackay, Cambridge (GB)

(21) Appl. No.: 17/792,535

(22) PCT Filed: Jan. 15, 2021
(86) PCT No.: PCT/GB2021/050087

§ 371 Date: Jul. 13, 2022

(30) Foreign Application Priority Data
Jan. 16, 2020 (GB) 2000649.0
Aug. 26, 2020 (GB) 2013386.4
Aug. 26, 2020 (GB) 2013387.2

(51) Int. Cl. G16B 20/20; G16B 40/20

(52) U.S. Cl. CPC G16B 20/20; G16B 40/20

(57) ABSTRACT

A screening system includes control circuitry that determines gene variants present in a compiled genome representative of a subject based on a difference between a reference genome and the compiled genome representative of the subject, and acquires phenotype information from an observation of the subject. The control circuitry further generates multi-dimensional data structure that includes the gene variants in respect of a first dimension, the phenotype information in respect of a second dimension; and a set of data samples in respect of a third dimension. The set of data samples includes the compiled genome sequence representative of the subject, and corresponding historical data samples of other subjects including their corresponding transcript information (for example, including phenotype information) of the other subjects and their gene variants. The control circuitry executes a gene variant interpretation using a correlation function to find phenotype-gene variant relationships based on the generated multi-dimensional data structure.

### TECHNICAL FIELD

The present disclosure relates generally to technologies relating to acquiring genomic data, and analysing the acquired genomic data, for example to reduce stochastic errors present in the data and to provide interpretations of the data; and more specifically, to screening systems and methods for processing acquired genomic information to provide corresponding gene variant interpretations.

### BACKGROUND

Advancements in medical and computational technologies have enabled genomic sequencing of biological samples and analysis of corresponding acquired sequenced genomic data to be implemented. An analysis of genetic material isolated from a biological sample involves a combination of many complex wet lab (in vitro) and in silico processes, wherein the processes start from acquiring a biological sample from a given individual. Contemporary sequencing technologies, for example next generation sequencing (NGS), are capable of sequencing long DNA molecules by converting them into smaller fragment molecules, sequencing the fragment molecules in amplified form to generate corresponding fragment sequences, and then piecing together the fragment sequences to generate a DNA read of the long DNA molecules. However, these aforementioned contemporary sequencing technologies are prone to stochastic errors.

Currently, there is significant amount of uncertainty in genomic data analysis of patients because of the inefficiencies and inaccuracies in current technology, systems, and methods. There are potentially several technical problems that cause such inefficiencies and inaccuracies in current technology, systems, and methods used in executing genomic data analysis and interpretation. Two primary problems for such inefficiencies and inaccuracies are data errors (e.g. stochastic distortions or noise in input data), and a nature of the input data itself. Moreover, even when genetic variants are determined in a DNA read, there arises stochastic uncertainty when seeking to classify the genetic variants as being benign (i.e. harmless) or being pathogenic (i.e. causing a given condition) due to missing information, unclear or conflicting information.

Moreover, data quality is crucial to any task that involves data analysis, and in particular in domains of machine learning and knowledge discovery, where there is a need to handle copious amounts of human genomic data which is inherently complex. Typically, techniques such as polymerase chain reaction (PCR) employed for DNA sequencing are often subject to various errors and ambiguities and the DNA sequencing data potentially comprises stochastic distortions. Moreover, in recent times, several computing tools have been developed for genomic data analysis and interpretation to obtain insights. Particularly, such computing tools often employ machine learning algorithms and artificial intelligence models to interpret the DNA related data. However, such computing tools require extensive training using labelled and/or unlabeled training data to train the machine learning algorithms, which is a time consuming and a resource-intensive process. Furthermore, such conventional artificial intelligence models (i.e. the prediction models) undergo complete retraining when a new input related to a previous input of a subject is fed into such conventional artificial intelligence or prediction models, which is undesirable. For example, many diagnostic test results and other information related to a subject typically are not available temporally simultaneously, and usually arrive as and when such diagnostic tests are conducted and when additional data related to a patient is available. Thus, the retraining in such cases not only creates a time lag in assessment of genomic data relating to a subject, but also increases an uncertainty in the genomic interpretation, with an associated risk of misinterpretation. For example, a time lag can occur between a given patient's blood samples being sequenced and there arising a discovery of new relevant scientific information potentially some years afterwards; for example, the new relevant scientific information concerns what a particular gene does when expressed. As a result of the time lag, a medical record for the given patient may potentially be marked as “unresolved” and the given patient's record not revisited later when more information becomes available.

Therefore, in light of the foregoing discussion, there exists a need to overcome the aforementioned drawbacks associated with conventional methods for processing, analyzing, or interpreting genomic data, to reduce effects of data errors and stochastic noise.

### SUMMARY

The present disclosure seeks to provide a screening system for processing genomic information for gene variant interpretation. The present disclosure also seeks to provide a screening method for (of) processing genomic information for providing gene variant interpretation. The present disclosure seeks to provide a solution to the existing problem of stochastic distortions or noise in data related to a genomic sequence arising from diverse sources that leads to incoherent gene variant interpretation of a given subject. An aim of the present disclosure is to provide a solution that overcomes at least partially the problems encountered in prior art, and to provide a screening system that effectively nullifies, or at least reduces, the effect of the stochastic distortions or noise in data acquired from diverse sources relating to a genomic sequence for achieving a more accurate and coherent analysis thereof.

In one aspect, the present disclosure provides a screening system comprising:
control circuitry that, when in operation:
   receives a plurality of genomic sequences of a plurality of genomic fragments of at least one biological sample from a subject that has been sequenced in a sequencing apparatus, wherein the plurality of genomic sequences includes stochastic errors and stochastic distortion;
   aligns the plurality of genomic sequences to a reference genome to generate from the aligned genomic sequences a compiled genome representative of the subject;
   determines one or more gene variants present in the compiled genome representative of the subject relative to the reference genome based on a difference between the reference genome and the compiled genome representative of the subject,
   acquires phenotype information from an observation of the subject, characterized in that the control circuitry further:
   generates a multi-dimensional data structure that includes:
      the one or more gene variants in respect of a first dimension;
      the phenotype information in respect of a second dimension; and
      a set of data samples in respect of a third dimension, wherein the set of data samples includes the one or more gene variants of the subject and their corresponding phenotype information, and corresponding historical data samples of other subjects including their one or more gene variants and their corresponding biological (for example, transcripts (for example, phenotype)) information;
   executes a gene variant interpretation using a correlation function to identify one or more phenotype-gene variant relationships based on the generated multi-dimensional data structure, wherein using the multi-dimensional data structure reduces a susceptibility of the gene variant interpretation to be affected by the stochastic errors and stochastic distortion.

In another aspect, an embodiment of the present disclosure provides a screening method for (namely, a method of) operating a screening system, characterized in that the method includes:
(i) using a control circuitry, to receive a plurality of genomic sequences of a plurality of genomic fragments of at least one biological sample from a subject that has been sequenced in a sequencing apparatus, wherein the plurality of genomic sequences includes stochastic errors and stochastic distortion;
(ii) aligning the plurality of genomic sequences to a reference genome to generate from the aligned genomic sequences a compiled genome representative of the subject;
(iii) determining one or more gene variants present in the compiled genome representative of the subject relative to the reference genome based on a difference between the reference genome and the compiled genome representative of the subject;
(iv) acquiring phenotype information from an observation of the subject;
(v) generating a multi-dimensional data structure that includes:
   the one or more gene variants in respect of a first dimension;
   the phenotype information in respect of a second dimension; and
   a set of data samples in respect of a third dimension, wherein the set of data samples includes the one or more gene variants representative of the subject and their corresponding phenotype information, and corresponding historical data samples of other subjects including their one or more gene variants and their corresponding biological (for example, phenotype) information;
(vi) executing a gene variant interpretation using a correlation function to find one or more phenotype-gene variant relationships based on the generated multi-dimensional data structure, wherein using the multi-dimensional data structure reduces a susceptibility of the gene variant interpretation to be affected by the stochastic errors and stochastic distortion.

In yet another aspect, an embodiment of the present disclosure provides a computer program product comprising a non-transitory computer-readable storage medium having computer-readable instructions stored thereon, the computer-readable instructions being executable by a computerized device comprising processing hardware to execute the aforementioned method.

Embodiments of the present disclosure substantially eliminate or at least partially address the aforementioned problems in the prior art, and enables generation of the first multi-dimensional data structure to reduce the stochastic errors, increase accuracy in gene variant interpretation, and reduce uncertainty in provisioning of decision support to assist a health care professional.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram that illustrates a network environment of a screening system, in accordance with an embodiment of the present disclosure;

FIG. 1B is a block diagram that illustrates a network environment of a screening system, in accordance with another exemplary embodiment of the present disclosure;

FIG. 3 is an illustration of an exemplary scenario for implementing a screening system for processing genomic information for generating a gene variant interpretation, in accordance with an exemplary embodiment of the present disclosure;

FIG. 4 is a schematic illustration of a matrix depicting phenotype-variant relationship probabilistically, associated with a screening system, in accordance with an embodiment of the present disclosure; and

FIG. 5 is a flowchart depicting steps of a screening method for (of) processing genomic information for generating gene variant interpretations, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible. Various embodiments of the present disclosure provide a system and a method for processing genomic information for generating gene variant interpretations.

In known conventional systems and methods, there are two primary problems, namely:
(i) data errors (e.g. stochastic distortions or noise in input data); and
(ii) a way in which the input data is designed and processed, which results in inaccuracies and misinterpretation of gene variants.

Other secondary problems include a problem of sporadic retraining of a conventional prediction model or system as and when new data related to a subject is available and fed into the conventional prediction model or system. Certain conventional systems are trained, for example, using artificial-intelligence (AI) tools, to process biological data (e.g. genomic information). Such AI tools are distinguished in that operation of their software is adaptively modified in operation by data processed via the AI tools; in contradistinction, conventional software tools, even when reconfigurable via control parameters, employ software that is not adaptively modified by data being processed through the conventional software tools. Some of these AI tools operate on a “black box” approach whose manner of internal working is often difficult to characterize and audit; for example, when black box neural networks are employed. Often, AI tools provide unpredictable results, for example when the AI tools are trained using sparse data, even if the manner of computation of such AI tools is auditable. Thus, such conventional systems, as a result, often fail to provide a coherent and meaningful analysis from the data arising from diverse sources, which increases an uncertainty of genomic interpretation and a risk of misinterpretation. In regard to such drawbacks associated with conventional systems, there is encountered potentially unreliable operation, or erratic operation, of such systems, which is undesirable.

Additionally, in certain scenarios, it may be required or may be useful, or both, to share genomic interpretation data and learnings from one system or institution to another system (or institution) for analysis purpose. However, due to the confidential nature of genomic and medical data of a given patient, the problem of sharing such data and learnings for analysis and gene therapy, respecting patient confidentiality as required by various national authorities/international regulations, increases manifold. Subsequently, a new conventional system needs to be trained independently for analysis of similar type of data from the diverse sources, which further increases cost of operation, time of training of AI-based tool used in such conventional system, and leads to duplication of human efforts required to train such conventional systems. In regard to such drawbacks associated with aforesaid conventional systems, there is encountered an increase in cost of gene variant interpretation.

In contrast to the conventional systems and methods, the disclosed screening system and method of the present disclosure provides a platform that uses a multi-dimensional data structure (i.e. an improved cross-related input data structure) to improve accuracy and reduce risk of misinterpretation of gene variants. The multi-dimensional data structure includes a set of data samples, which includes a compiled genome sequence representative of the subject, and corresponding historical data samples of other subjects including their corresponding phenotype information of the other subjects and their one or more gene variants. Such a multi-dimensional data structure reduces sensitivity of the gene variant interpretation to the stochastic errors and stochastic distortion, and thus the risk of misinterpretation of gene variants is significantly reduced.

Moreover, the disclosed screening system of the present disclosure reduces the risk of misinterpretation of gene variants and enables an incremental reduction of uncertainty in gene variant interpretation to find one or more phenotype-gene variant relationships, for example, upon acquiring new input related to the subject. The disclosed screening system of the present disclosure further effectively nullifies an effect of the stochastic distortions or noise in input data that is used for the gene variant interpretation, and thus the risk of misinterpretation of gene variants is significantly reduced. Moreover, making the system independent of wholesale re-training (namely, training on all previous data as well as new data) further enhances computational efficiency of the system by substantially increasing its speed of operation, and reducing a chance of faulty training arising, which may have practical life-saving implications for the subject. In other words, the screening system utilizes a model that is incrementally trained; the model is trained on a given day, and then thereafter the model is adjusted, (namely retrained) only on new data that are added subsequently. Such retraining is beneficially implemented periodically, namely in a manner of “incremental learning”.

Furthermore, making the system independent of re-training also decreases data storage requirements for operation of the screening system. Furthermore, the disclosed screening system of the present disclosure is comparatively less computer intensive and requires less data storage space at the time of processing the genomic data. Consequently, random access memory is available for performing other tasks.

Throughout the present disclosure, the term “screening system” refers to a system for processing and analyzing biological data to derive insights therefrom. The screening system may also refer to control instruments, control circuitries and/or data processing systems for operation thereof and to obtain results relating to the biological data. Notably, the screening system substantially reduces stochastic errors and stochastic distortion when determining insights from the biological data and providing a higher accuracy when deducing results derived from different portions of genomic sequences (e.g. gene sequences and variants thereof) of subjects.

The screening system comprises the control circuitry. The control circuitry refers to a computational element that is operable to respond to and processes instructions that drive the screening system. Optionally, the control circuitry includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “control circuitry” may refer to one or more individual processors, processing devices, a part of an artificial intelligence (AI) system, and various elements associated with the screening system.

The control circuitry, when in operation, receives a plurality of genomic sequences of a plurality of genomic fragments of at least one biological sample from a subject that has been sequenced in a sequencing apparatus, wherein the plurality of genomic sequences includes stochastic errors and stochastic distortion; optionally, the sequencing apparatus is implemented as proprietary sequencing apparatus, for example as manufactured by Illumina® Corp. or Qiagen® Corp. Firstly, the at least one biological sample is isolated from the subject. The biological sample of the subject refers to a laboratory specimen taken by sampling under controlled environments, that is, gathered matter of a medical subject's tissue, fluid, or other material derived from the subject. Examples of the biological sample include, but are not limited to, blood, throat swabs, sputum, saliva, surgical drain fluids, Chorionic villus sampling (CVS), tissue biopsies, amniotic fluid, or sample of foetus, such as cell free foetal DNA. The sample of foetus is used to identify variations in prenatal testing. For example, the detection of early-infantile epileptic encephalopathy (EIEE) may be performed by using the sample of foetus. The EIEE is a rare neurological disorder characterized by seizures. It is observed that epilepsy, in a significant percentage of children, is wrongly identified and treated as gastro-intestinal disorders.

According to an embodiment, the biological sample is processed in vitro using a wet-laboratory arrangement to extract genetic material from the biological sample, and prepared for sequencing in the sequencing apparatus. As used herein, the term “wet-laboratory arrangement” refers to a facility, clinic and/or a setup of instruments to collect and process the biological sample for extraction, amplification, enrichment, and/or processing of genetic material extracted from biological sample. Herein, the instruments, equipment, and/or devices may include, but are not limited to, centrifuges, spectrophotometers, PCR, RT-PCR, High-Throughput-Screening (HTS) systems, Microarray systems, Ultrasound, and genetic analysers. The wet-laboratory arrangement processes the biological sample and obtains DNA fragments. Specifically, DNA fragments present in biological sample are amplified and sequenced using known sequencing techniques.

In an example, in order to execute sequencing (e.g. next generation sequencing), an input sample, such as DNA, of the subject is isolated from the biological sample of subject. For example, after sampling blood, a small amount of DNA is isolated from the sampled blood. The quantity of isolated DNA is insufficient for sequencing library preparation. Therefore, the input sample is then fragmented into short sections. The length of these sections is optionally same, for example, about 300 base pairs, optionally in a range of 100 to 250 base pairs. The length optionally also depends on a type of sequencing machine used or a type of experiment to be conducted. In some cases where the length of DNA sections is relatively longer, for example longer than 250 base pairs, the fragments are ligated with generic adaptors (i.e. small piece of known DNA located at the read extremities) and annealed to a glass slide using the adaptors (e.g. in Illumina®-based sequencing). In some cases, mRNA transcripts are isolated which correspond to the coding regions of functional genes, for example in exome sequencing.

According to an embodiment, the sequencing apparatus is configured to, namely is operable to, execute sequencing of the plurality of genomic fragments. In an example, the plurality of genomic fragments are potentially a plurality of complementary deoxyribonucleic acid (cDNA) fragment molecules that are sequenced concurrently in a next generation sequencing (NGS) (i.e. short reads sequencing known in the art) to generate the plurality of genomic sequences. Notably, sequencing, for example, DNA sequencing, is a process of determining a sequence of nucleotides in a given section of DNA. Moreover, the plurality of genomic sequences obtained employing techniques such as polymerase chain reaction (PCR) and NGS, often comprise stochastic errors resulting from the amplification and sequencing process. Beneficially, the screening system described herein provides significantly more accurate results despite the stochastic errors being present in the plurality of genomic sequences.

The control circuitry, when in operation, aligns the plurality of genomic sequences to a reference genome to generate from the aligned genomic sequences a compiled genome representative of the subject. The control circuitry is further configured to, namely operable to, compare the plurality of genomic sequences with the reference genome in the alignment. In an example, the reference genome is potentially a latest version of genome build assembly (e.g. GRCh38/hg38 human genome build assembly). Alternatively, the reference genome of an animal species or genus may be used in case the subject is same animal of same species (or genus). Thus, the sequence readout data for each fragment of the plurality of genomic fragments that is the plurality of genomic sequences is pieced together to recreate a final DNA readout which is the compiled genome representative of the subject; when piecing the sequence readout data together, there is overlap and ambiguity that is manifest as sequencing uncertainty in the final DNA readout data. In an example, the alignment is performed via a graphical user interface with the capability of high zoom in resolution so that the alignment of the base pairs is verifiable. Such alignment is performed, for example, manually via a graphical user interface of a computing system.

The control circuitry, when in operation, determines one or more gene variants present in the compiled genome representative of the subject relative to the reference genome based on a difference between the reference genome and the compiled genome representative of the subject. It will be appreciated that a majority of the DNA of a subject is same across all humans. The differences may indicate a plurality of gene variants responsible for different traits in the subject. Notably, some of the plurality of gene variants may also be responsible for occurrence of a disease in the subject. The difference between the reference genome and the compiled genome representative of the subject enables to identify meaningful variation in an individual's genome sequence to distinguish what is healthy from what is potentially pathological. Examples of the one or more gene variants determined include, but are not limited to, copy number variants (CNVs), indels, single nucleotide variants (SNV), and other mutations responsible for rare genetic diseases. In other words, the final DNA readout of the given subject (after compilation) is then compared with the reference genome, usually an aggregate of many DNA readouts, and then differences between the final DNA readout of the given individual and the reference genome are then identified. It is in these differences (i.e. the gene variants) in which rare disease may be present in comparison to the reference genome that corresponds to a heathy individual without the rare diseases.

Optionally, the screening system is configured to, namely is operable to, generate a graphical representation of the alignment on the graphical user interface of the screening system. The control circuitry is further configured to, namely is operable to, determine locations of each of the determined one or more gene variants. Optionally, the determined one or more gene variants or other genes are annotated (or tagged) by using the graphical user interface. The annotations are generated automatically or semi-automatically (namely, is user-assisted or allows for user-input for editing). The annotations are editable via the graphical user interface. Examples of the annotations include, but are not limited to, gene(s) loci, locations of coding regions (e.g. exons) in the portion of the genomic sequence, known functions of genes, or gene variants (annotations of detected CNVs, SNVs, indels, etc), adding gene variant unique identifiers, gene variant names, zygosity information, parental information, understanding of gene or gene variants retrieved from known and credible literature sources (e.g. research publications), or a relation to a known phenotype. Generally, such annotation is made using an explanatory note or comment at the location of the one or more gene variant (e.g. an additional data point or field).

Optionally, the compiled genome representative of the subject is also aligned to other one or more known genetic variant sequences to determine further if any are missed, or to fine-tune the determined one or more gene variants, or both. For example, the one or more known genetic variant sequences may be obtained, for example, from genomic databanks, public scientific databases, databases of research organizations (e.g. Database of Genomic Variants (DGV), Online Mendelian Inheritance in Man (OMIM), MORBID, DECIPHER), research literature (e.g. PubMed literature), and other supporting information, and so forth. Optionally, heteroplasmic variants that contribute to phenotype (e.g. a disease) are potentially detected in the compiled genome representative of the subject. Moreover, the control circuitry is configured, namely is operable, to detect mosaic variants, and whether a mutation is an inherited mutation or a de novo mutation. The different gene variants are then tagged as per the type of variant (i.e. type of mutation) at a corresponding site on the complied genome that is aligned across the reference genome and visualized via the graphical user interface. Based on the detection of additional gene variants from the alignment to one or more known genetic variant sequences, additional annotations corresponding to such detection may be auto-populated (or manually tagged in some cases) on the graphical user interface.

In an example, a gene name (e.g. ‘BICD2’ gene) and online Mendelian Inheritance in Man (OMIM) identifier (ID) (e.g. ‘609797’) are assigned to a gene variant. OMIM include publicly available information on known mendelian disorders of about 15,000 genes, which is periodically updated and contain the relationship between phenotype and genotype. ‘MORBID ID’ (e.g. 615290) is also assigned. A ‘MORBID ID’ is indicative of a chart or diagram of diseases and the chromosomal location of genes the diseases are associated therewith. The morbid map is provided in the OMIM knowledgebase, listing chromosomes and the genes mapped to specific sites on those chromosomes. Known conditions associated with the gene (e.g. the BICD2) gene is also annotated (e.g. conditions: Proximal spinal muscular atrophy with autosomal-dominant inheritance). Thus, the datapoint ‘autosomal dominant’ which is a good indicator of the conditions for preparation of the aforementioned multi-dimensional data structure (described later below). Optionally, a HI score (e.g. 0.176) is also assigned to each gene that indicates zygosity of the gene. Furthermore, based on comparisons and determination of various types of mutations (e.g. missense variant, copy number variants, and the like) are determined and added as annotations to the gene sequence datapoint. A genotype (e.g. heterozygous, homozygous, and the like) datapoint is also assigned. Furthermore, other than comparison with known variants, curated variants are also used for comparison to determine information for variants. Other accessory information, for example, The Human Phenotype Ontology (HPO) terms are assigned which provides a standardized way to represent phenotypic abnormalities encountered in human disease. It is also automatically retrieved, if the gene sequence (e.g. BICD2) is previously reported as pathogenic, and what prior information is available in this regard. Furthermore, if the gene is found to be pathogenic then, what is the contribution of the gene variant to phenotype is also ascertained. For example, if the contribution of the gene variant is partial, full, uncertain, or none. Thus, various other datapoints are added as supplementary or supporting information, e.g. it is detected upon alignment of the complied genome representative of the subject with parental gene sequences of the same gene, whether the mutation is inherited or de novo.

The control circuitry, when in operation, acquires phenotype information from an observation of the subject. For example, a healthcare professional may assess the subject for potential diseases or distinguishing traits. Any condition or disorder may be noted, and assigned phenotype codes based on observed characteristics of the subject. Alternatively, ICD codes (International Classification of Diseases) codes are assigned and phenotype codes are then derived from the ICD codes usually provided by the healthcare professional. The phenotype codes may be assigned in accordance with a publicly known database, known as “Monarch initiative”, which integrates a variety of externally curated data sources, primarily focused on genotype-phenotype and disease-phenotype associations. Such phenotype codes that corresponds to observed characteristics of the subject (e.g. a patient suffering from some illness or disorder), is referred to as phenotype information, and stored in a database, from which the phenotype information is acquired to check if the observed phenotype is as a result of any gene variant by the screening system.

The control circuitry, when in operation, further generates a multi-dimensional data structure that includes:
the one or more gene variants in respect of a first dimension;
the phenotype information in respect of a second dimension; and
a set of data samples in respect of a third dimension, wherein the set of data samples includes the one or more gene variants of the subject and their corresponding phenotype information, and corresponding historical data samples of other subjects including their one or more gene variants and their corresponding biological (for example, phenotype) information.

Optionally, the multi-dimensional data structure can have more than three dimensions, for example an additional dimension of ethnicity of the set of data samples, an additional dimension of ionizing radiation exposure history, and so forth.

The control circuitry is configured, namely is operable, to generate the multi-dimensional data structure. The control circuitry is further configured to generate the first multi-dimensional data structure based on a combination of the determined one or more gene variants, the phenotype information, and the set of data samples. The determined one or more gene variants refers to gene variants in the compiled genome representative of the subject identified based on one or more of: the alignment of the compiled genomic sequence of the subject with reference genome, alignment to publicly available gene variant databases, and gene variant detection algorithms of the screening system. The phenotype information refers to the acquired phenotype information that may be stored in respect of the second dimension and vis-à-vis the determined one or more gene variants to facilitate finding of a pattern or relationship among one or more gene variants and the acquired phenotype information by the screening system in a downstream operation, such as gene variant interpretation (discussed later below). The historical data samples of other subjects including their corresponding phenotype information of the other subjects and their one or more gene variants refers to previously determined and validated gene variants with known phenotype information of the other subjects. The data elements in the three dimensions, first, second, and the third dimension are arranged in a relational and common form to enable efficient and accurate analysis multi-dimensional data elements in the multi-dimensional data structure.

Additionally, and optionally, the data from diverse sources usually vary in nature owing to, for example, different terminologies used, different emphasis, and incoherent output of the diverse sources. Subsequently, in the multi-dimensional data structure, the data elements in the first, second, and the third dimension are potentially stored in a multi-dimensional array, and converted to a common machine-readable format that is parsable by a computing machine, particularly, an artificial-intelligence (AI) based system. Beneficially, the conversion of the various data elements (i.e. data values of various data fields) in the common format enables efficient access and modification of the data elements.

Optionally, the control circuitry is configured, namely is operable, to detect the deviations in the data elements of the multi-dimensional data structure. The deviations are potentially detected if there a mismatch in data elements between any two dimensions of multi-dimensional data structure. For example, a boundary of a sequence of the determined gene variant may not coincide with a boundary of a sequence derived from historical information of one or more gene variants of other subjects in the set of data samples. In an example, a risk of a child inheriting a disorder having parents with gene responsible for the same disorder is potentially more. Thus, one data element potentially complements or deviates from another data element when the correlation and associations are made. Such potential deviations and initial correlation in the data elements potentially enables self-correction of erroneous or inconsistent datapoints (i.e. by filtering or flagging of inconsistent datapoints in the first multi-dimensional data structure).

In an example, a likelihood of a mutation within a region, a likelihood of an error during amplification and/or sequencing of DNA fragments, or variations in a phenotype influenced by factors such as diet, climate, exposure to chemicals or ionizing radiation, illness and so forth, may be determined. In an example, certain information for external sources, such as information received from abnormality scans performed during pregnancy to ensure a healthy development of foetus, may indicate a phenotype or manifestation of a genetic anomaly. Such information when correlated may indicate a phenotype versus gene variant statistical relationship, and also enable detection of the deviation in the data elements from multi-dimensional perspective.

In another example, a black list and a white list of gene variants are prestored in a database server of the screening system. The black list and the white list of gene variants are potentially part of the set of data samples. Variants added to the blacklist are not displayed in gene variant table (or list) during annotations regardless of any filters applied. This provides a mechanism for filtering out known off target variants in a gene of interest, or known sequencing artefacts (sequencing data errors), thereby contributing in the self-correcting property of the first multi-dimensional data structure. The white list curated lists contain previously curated data and take precedence over the blacklist. Thus, when gene panels are assigned to a subject, the curated list filters are exclusively applied to genes in the areas of interest defined by the gene panels. For example, a white listed gene is not shown if the gene is outside the area of interest. Targeted gene sequencing panels are useful tools for analysing specific mutations in a given data sample. Focused gene panels contain a select set of genes or gene regions that have known or suspected associations with the disease or phenotype under study, and thus the white listed gene is not shown if the gene is outside the area of interest. This saves storage space in the data memory device of the screening system.

Optionally, additional datapoints or annotations related to variant effect predictor (VEP) consequence or a type of gene variant is also added for a determined gene variant as annotation in the multi-dimensional data structure. For example, the type of various gene variants includes, but is not limited to, transcript ablation, splice donor variant, splice acceptor variant, stop gained, frameshift variant, start lost, initiator codon variant, transcript amplification, inframe insertion, inframe deletion, missense variant, protein altering variant, splice region variant, incomplete terminal codon variant, synonymous variant, coding sequence variant, mature miRNA variant, 5 prime UTR variant, 3 prime UTR variant, non-coding transcript variant, intron variant, upstream gene variant, downstream gene variant, transcription factor (TF) binding site variant, regulatory region ablation, transcription factor binding sites (TFBS) ablation, and the like. Such datapoints are indicator of how likely a type of gene variant will have a contribution to phenotype. This further facilitates in determining strength of influence of a gene variant in the manifestation of an observed phenotype at the time of the gene variant interpretation. Further, population data (e.g. African, south Asian, Finnish, American, African American etc.) are also added as additional annotations in the multi-dimensional data structure, which is useful in downstream processing of the data elements in the multi-dimensional structure.

According to an embodiment, the screening system processes, when in operation, the one or more gene variants present in the compiled genome representative of the subject relative to the reference genome based to reduce stochastic errors due to at least one of: indels, copy number variations (CNVs), substantial palindromes, incorrectly identified or mis-classified phenotypes. Optionally, the different data points stored in the multi-dimensional data structure are related to each other, and collectively augments understanding of the compiled genome representative of the subject, and reduces misapprehension so as to remove errors and inconsistencies therefrom. Furthermore, a potential ripple effect of the stochastic errors and stochastic distortion in the multi-dimensional data structure is reduced in all subsequent operations that use the multi-dimensional data structure (e.g. multi-dimensional data elements stored in the multi-dimensional data structure). Beneficially, such removal of the errors and the inconsistencies from the multi-dimensional data-structure enhances reliability of the multi-dimensional data structure for subsequent operations and further enhances reliability of output produced by employing such multi-dimensional data structure.

The control circuitry, when in operation, executes a gene variant interpretation using a correlation function to find one or more phenotype-gene variant relationships based on the generated multi-dimensional data structure, wherein using the multi-dimensional data structure reduces a sensitivity of the gene variant interpretation to the stochastic errors and stochastic distortion. The control circuitry is configured, namely operable, to execute the gene variant interpretation based on the input of the data elements in the first multi-dimensional data structure. Notably, “gene variant interpretation” refers to a process of explicating a pattern or correlation between the acquired phenotype information (observed characteristics of the subject) and a potential genetic cause (e.g. a gene variant) at least one phenotype in the phenotype information. The correlation function is a function that finds a statistical correlation between random variables (e.g. data elements in this case) in the multi-dimensional data structure. The identified statistical correlation may be in the form of latent variables that are embedded within the model in relation to the multi-dimensional data structure. The execution of the correlation function in relation to the latent variables generates the later described one or more Bayesian mappings. Examples of the correlation function may correspond to one or more later described adaptive artificial intelligence (AI) or machine learning (ML) arrangements to generate the one or more Bayesian mappings. As an option, the correlation functions may further include but are not limited to one or more matrix factorization algorithms as described. Based on historical information, such as the historical data samples of other subjects including their corresponding phenotype information of the other subjects and their one or more gene variants, a check is made whether or not one or more phenotype codes that represents phenotype information of the subject are caused by one or a set of gene variants that are previously determined by the screening system and stored in the multi-dimensional data structure. The correlation function is used to find such one or more phenotype-gene variant relationships for the subject. Additionally, and optionally, the gene variant interpretation further enables identification of disease susceptibility in the subject, reaction of the subject towards a given drug, and so forth. According to an embodiment, the control circuitry is configured, namely is operable, to store the gene variant interpretation in a database server. The database server may be hardware, software, firmware and/or any combination thereof. The database server includes any data storage software and systems, for example, a relational database.

According to an embodiment, the screening system is configured, namely is operable, to generate a graphical representation of the one or more phenotype-gene variant relationships for user-editing and adjustment on a graphical user interface, wherein the graphical representation also provides a strength of correlation. The one or more phenotype-gene variant relationships are displayed on the graphical user interface, and such graphical representation is editable. The screening system provides a clinical expert (i.e. a user of the screening system) the graphical representation of the one or more phenotype-gene variant relationships so that validation can be done, and if any doubt occurs, such results can be cross-related with historical reports and the basis of output of such results can be traced, and audited for confirmation, via the graphical user interface.

According to an embodiment, the screening system generates one or more Bayesian mappings describing one or more phenotype-gene variant relationships that have a probability that exceeds one or more threshold criteria. The Bayesian mappings employs statistical rules in accordance with Bayes principle (e.g. Bayesian inference rules) to describe one or more phenotype-gene variant relationships for the subject that have a probability that exceeds one or more threshold criteria. Threshold criteria may further specify or dictate boundaries to which determines the phenotype-gene variant relationships. The one or more threshold criteria are prespecified to meet a specified accuracy requirement in the one or more phenotype-gene variant relationships. In an example, the one or more Bayesian mappings may employ a Bayes factor to describe the one or more phenotype-gene variant relationships. In another example, the Bayesian mappings may be a combined representation of each of the probability associated with the phenotypic categories (such as benign, likely benign, likely pathogenic, and pathogenic) for the interested variant for a patient. This combined representation may be in the form of a histogram or other graphical representation suitable for displaying the resultant probabilities. The probabilities may be similarly viewed as the likelihood of a phenotypic category for a gene variant given the multi-dimensional data structure. For instance, the Bayes factor potentially indicates a likelihood of a phenotype in the acquired phenotype information of the subject as a result of a determined gene variant in the subject in the multi-dimensional data structure. It is likely that instead of a single gene variant, two or more gene variants are responsible for the manifested phenotype in the subject. The Bayes mappings may indicate a strength of influence of each gene variant of the two or more gene variants in the manifestation of the phenotype in the subject. As more evidence is obtained from the data elements, such as the multi-dimensional data structure (e.g. the historical data samples of other subjects including corresponding phenotype information of the other subjects and their one or more gene variants) and/or new data elements as and when obtained for the subject and stored in the corresponding dimension of the multi-dimensional data structure, the likelihood of the cause of the phenotype in the acquired phenotype information of the subject as a result of one or more determined gene variant in the subject increases. Optionally, a directed acrylic graph (DAG) may be used to define association and relations between a gene variant and corresponding phenotype. According to an embodiment, the screening system employs an adaptive artificial intelligence (AI) or machine learning (ML) arrangement to generate the one or more Bayesian mappings. Notably, the term “adaptive artificial intelligence (AI)” or “machine learning arrangement” refers to AI-enabled circuitry or adaptive software that employs one or more neural network models or Bayesian network models to generate an output, without being explicitly programmed therefor. Specifically, the adaptive artificial intelligence or machine learning arrangement is employed to acquire information and a set of rules, the set of rules are used to process the acquired information from the multi-dimensional data structure so as to generate an output. The output generated further undergoes correction to achieve a desired level of reliability and efficiency. Typically, examples of the different types of neural network models or the Bayesian network models include, but are not limited to: supervised learning model, unsupervised learning model, a semi-supervised learning model, a conditional probability and directed acrylic graph-based learning model, and reinforcement machine learning model. For example, an error is computed at an output layer of the adaptive artificial intelligence arrangement based on the accuracy of each output in a training phase. Specifically, the term “error” refers to a deviation from of a generated output from a desired output (expected output). In an example implementation, the error is measured in terms of percentage. Therefore, the computed error is fed (namely, back propagated) thereto, so as to train the adaptive artificial intelligence arrangement. Beneficially, Bayesian mappings to find gene variant-phenotypic relationships are learned based on the training.

More specifically, datapoints that correspond to the multi-dimensional data structure may be annotated during the training of the adaptive AI or ML arrangement. That is, the annotated datapoints (i.e. variant annotations) may be used for the derivation or generation of latent variables. These latent variables are associated with the adaptive AI or ML arrangement and correspond to the Bayesian mapping. The latent variables capture the abstract notion of the pathogenic categories to which an assessment of a gene of interest may be determined.

Further, the adaptive artificial intelligence arrangement may employ various types of training data or annotated data or datapoints. These data include but are not limited to the dataset associated with Patient ID, Patient Phenotype, Variant ID, Pathogenic Metric, and side information. Patient ID may be unique identifiers for each patient. Patient Phenotype are phenotypes observed for the patients and may be presented as Human Phenotype Ontology (HPO) terms. One example of an HPO term is HP: 0000729 for patients with Autistic behaviour phenotype; and another example is HP: 000986 for patients with Limb undergrowth phenotype. Variant ID may be unique for each variant. Variant ID may present features that are concatenated and separated by underscore(s). For example, Variant ID 2_1765342_C_T_NM_00193456 uniquely identifies the variant on chromosome 2, starting at the base pair position 1765342, involving the mutation C>T on the transcript NM_00193456. Here, the Variant ID 2_1765342_C_T_NM_00193456 identifies the Chromosome, Start, Ref allele, Alt allele, and Transcript ID. Pathogenic Metric may be represented by the pathogenicity level of the variant as defined by American College of Medical Genetics (ACMG). For example, there may be a Pathogenic Metric B for Benign, LB for Likely Benign, LP for Likely Pathogenic, P for Pathogenic, and VUS for Uncertain Significance. These may be alternative training labels, for example, adapted to the matrix factorization algorithm. The side information may be presented as variant's annotations used in the cosine similarity or organized in any suitable format used in a supervised learning framework.

The training data or annotated data are used for training the Pathogenicity Model to assess and compute the probability distribution for a gene variant in order to assess the pathogenicity of a variant for a patient. Specifically, the training data or annotated data may be organized in computer-readable formats that include but are not limited to a real number, binary, categorical, identifier, lists, and strings formats that are suitable for processing with one or more models, frameworks, algorithms, techniques, and methodologies here described.

A practical example of training data or annotated data in relation to the types of training data is shown in Table 1 below. The table also shows features associated with the side information for a given variant. For example, one feature may be the maximum allele frequency for the patient; another feature may be the non-synonymous amino acid change in a functional protein domain for the same patient. Each feature (of features 1 to 11) is presented in the table in relation to the Patient ID, Patient Phenotype, Variant ID, and Pathogenic Metric. Other presentation of training data include the example in table 1 but are not limited to this example. Training data may be presented and organised in relation to the model, framework, algorithm, techniques, or methodology applied. The training data may be presented to accommodate as inputs for training the Pathogenicity Model as described herein.

TABLE 1

| Patient | Patient | | Pathogenic | Feature | Feature | Feature | Feature |
|---|---|---|---|---|---|---|---|
| ID | Pheotypes | Variant ID | metric | 1 | 2 | 3 | 4 |
| 1 | HP:000164 | 7_1506460 | B | 0 | 3.95 | | |
| 1 | HP:000164 | 11_768348 | LB | 0.005277 | −0.163 | | |
| 1 | HP:000164 | 16_579939 | P | 0.000124 | −1.5 | 0.03 | 0.001013 |
| 2 | HP:000047 | 12_485164 | VUS | 0.218986 | 4.38 | 0.036 | 0.004091 |
| 3 | HP:000070 | 8_1007791 | B | 0.008287 | −2.49 | | |
| 3 | HP:000070 | 8_5553922 | LP | 0 | 4.2 | | |
| 3 | HP:000070 | 10_897208 | P | 0 | 4.39 | | |
| 4 | HP:000124 | 9_1194602 | B | 0 | 4.43 | 0.67 | 0.12 |
| 5 | HP:000047 | 3_3865144 | B | 0.006742 | 0.209 | 0.001 | 0.23 |
| 5 | HP:000047 | 6_4268955 | P | 6.06E−05 | 5.78 | | |
| 6 | HP:000048 | 5_8999044 | VUS | 0.003192 | 5.81 | | |
| 6 | HP:000048 | 5_7094599 | VUS | 0.00015 | 3.84 | 0.45 | 0.98 |
| 7 | HP:000058 | 2_1795474 | LB | 0.01105 | −3.98 | | |
| 7 | HP:000058 | 18_485934 | P | 1.00E−04 | 5.49 | 0.34 | 0.109 |
| 8 | HP:000194 | 9_1171857 | VUS | 0.009235 | 4.41 | | |
| 8 | HP:000194 | 11_663347 | B | 0.000539 | −1 | 0.001 | 0.876 |
| 8 | HP:000194 | X_4907497 | LB | 0 | 4.73 | | |
| 9 | HP:000194 | 3_1506583 | VUS | 0.001079 | 0.649 | 0.762 | 0.999956 |
| 9 | HP:000194 | 6_1372193 | LP | 0 | 5.96 | | |
| 9 | HP:000194 | 10_735581 | B | 0.005642 | 4.63 | | |
| 9 | HP:000194 | 17_364935 | LP | 0.005394 | 3.1 | | |
| 10 | HP:000194 | 10_735376 | B | 0.000458 | −11 | | |
| 11 | HP:000150 | 4_3634519 | LB | 0 | 2.58 | 0.987 | 0.567 |
| 11 | HP:000150 | 15_784016 | P | 0.0032 | −7.53 | 0.26 | 0.02 |
| 12 | HP:000047 | 11_119212 | VUS | 0.008287 | −6.19 | 0.4 | 0.6 |
| 13 | HP:000070 | 2_2024980 | B | 0.006272 | 1.46 | 0.6 | 0.24 |
| Patient | Feature | Feature | Feature | Feature | Feature | Feature | Feature |
| ID | 5 | 6 | 7 | 8 | 9 | 10 | 11 |
| 1 | frameshift_variant | | | 0.697 | | | 0 |
| 1 | missense_ | 0.002 | 0.64 | 0.208 | 5 | | 0 |
| 1 | splice_region_variant | | | 0.68 | | | 1 |
| 2 | intron_ variant | | | 0.21 | | | 1 |
| 3 | synonymous_variant | | | 0.277 | | Likely beni [symbol] | 0 |
| 3 | frameshift_ variant | | | 0.298 | | | 0 |
| 3 | stop_gained | | | | | Pathogenic | 0 |
| 4 | synonymous_variant | | | 0.192 | | | 0 |
| 5 | synonymous_variant | | | 0.242 | | Likely beni [symbol] | 0 |
| 5 | missense_ | 0.203 | 0.04 | 0.346 | 43 | | 0 |
| 6 | missense_ | 0.018 | | 0.066 | 29 | Likely beni [symbol] | 0 |
| 6 | missense_ | 0.037 | 0.05 | 0.032 | 43 | | 0 |
| 7 | synonymous_variant | | | 0.352 | | Likely beni [symbol] | 0 |
| 7 | missense_ | 0.912 | 0.04 | 1 | 32 | Uncertain [symbol] | 0 |
| 8 | missense_ | 0.88 | | 0.248 | 98 | Likely beni [symbol] | 0 |
| 8 | synonymous_variant | | | 0.109 | | | 0 |
| 8 | stop_gained | | | 0.231 | | | 0 |
| 9 | splice_acceptor_variant | | | 0.166 | | Uncertain [symbol] | 1 |
| 9 | missense_ | 0.905 | 0.13 | 0.096 | 22 | | 0 |
| 9 | synonymous_variant | | | 0.274 | | Likely beni [symbol] | 0 |
| 9 | missense_ | 0.052 | 0.13 | 0.07 | 43 | Uncertain [symbol] | 0 |
| 10 | missense_variant | | | 0.274 | 23 | | 0 |
| 11 | missense_ | 0.026 | 0.46 | | 145 | | 0 |

| | | | | |
|---|---|---|---|---|
| 11 | synonymous_variant | 0.313 | | 0 |
| 12 | synonymous_variant | 0.158 | Likely beni [symbol] | 0 |
| 13 | synonymous_variant | 0.073 | Likely beni [symbol] | 0 |

[symbol]

indicates data missing or illegible when filed

In another example, the adaptive AI or ML arrangements used to derive the latent variables may include one or more matrix factorization algorithms, but are not limited to Latent Dirichlet Allocation, Non-Negative Matrix Factorization, Bayesian and non-Bayesian Probabilistic Matrix Factorization, Principal Component Analysis, Neural Network Matrix Factorization, and the like. These algorithms may be used in applications such as collaborative filtering and recommender system applications, where the aim is to model relational data associated with these applications. Other adaptive AI or ML arrangements may include “curve fitting” algorithms such as linear regression with different penalties (i.e. LASSO, RIDGE, Elastic Net).

According to an embodiment, the control circuitry is configured, namely is operable, to associate the one or more generated Bayesian mappings describing one or more phenotype-gene variant relationships with a secondary database of historical medical reports to identify one or more historical medical reports that are related in subject matter to the one or more generated Bayesian mappings, and to present the identified one or more historical medical reports as a graphical list on the graphical user interface. The control circuitry is further configured to control the display of the graphical user interface on a display screen of the screening system. The identified one or more historical medical reports of the subject that are identified to be relevant to the one or more phenotype-gene variant relationships are displayed on the graphical user interface. In an example, this allows to link and verify the one or more phenotype-gene variant relationships vis-à-vis actual medical reports that also indicates same phenotype or genetic anomaly.

According to an embodiment, the screening system, when in operation, uses the identified one or more generated Bayesian mappings and the identified one or more historical medical reports to provide decision support information in respect of the subject. The decision support information is generated and displayed via the graphical user interface. The decision support information is indicative of a likelihood of the phenotype (e.g. a rare disease) due to a specific gene variant detected in the compiled genome of the subject. Optionally, the decision support information is generated and displayed on selection of a decision support mode. The decision support information for the subject, and other data, for example, the one or more gene variant-phenotype relationships obtained by the Bayesian mappings, are then added as further learnings in the screening system, thus the screening system becomes more robust over time. Alternatively stated, the corpus of data of new individuals grows with time and aggregation reduces uncertainty.

Optionally, the control circuitry is configured to render the graphical user interface that includes the results (i.e. the identified one or more generated Bayesian mappings describing the one or more gene variant-phenotype relationships) and evidence (e.g. the one or more historical medical reports) of the determined gene variant-phenotypic relationships, which is outputted with a confidence score specific for the subject. The confidence score indicates a percentage probability (i.e. the first probability e.g. 98% probability that is greater than the preset threshold of, for example, X percent, such as 90%) of the gene variant-phenotypic relationship, which assists a physician to conveniently asses presence or absence of a disease (i.e. manifested phenotype) with certainty. For example, the control circuitry is further configured to generate a confidence score that indicates a probability of a determined gene variant to be associated with the phenotype based on the executed gene variant interpretation. Specifically, the confidence score characterizes a certainty for the associations, e.g. a gene variant-phenotype relation, as described above. Optionally, the confidence score is a numerical value, an alphabetical grade, a rating, a ranking, a percentage, and so forth. Optionally, the confidence score is generated as a matrix. In an example, the confidence score that is indicative of the probability is defined between ‘0’ and ‘100’. In such case, ‘0’ indicates that an association is ‘certainly incorrect’ and ‘100’ indicates that an association is ‘certainly correct’.

According to an embodiment, a sequence of events that causes the output of the decision support information is linked with actual quantitative and qualitative information (e.g. medical reports and phenotype information from actual observation of subject) to enable scrutiny of the decision-making process. Subsequently, controlling the display of the decision-making process by the screening system enhances transparency of output generated by the screening system (including operation of the artificial intelligence or machine learning arrangement for the Bayesian mappings). Beneficially, displaying the decision-making process allows a user of the system to logically comprehend a behaviour of starting from the input, processing decisions, up to output. For example, from the input of the data elements of the multi-dimensional data structure related to the subject to the output of the decision support information, all the logical sequence of events is potentially visualizable via the graphical user interface. This enhances the authenticity and credibility of the screening system so that the results can be conveniently used by the physician for various applications.

According to an embodiment, the control circuitry is configured, namely in operable, to augment a prior input of the data elements in the multi-dimensional data structure by a new input (e.g. as new batches of data arrive from further observation by clinical experts or genetic tests or historical data of other subjects in the set of data samples) in the screening system. The new input is treated as the supplementary input to augment the prior input instead of entirely a new input. Therefore, the screening system does not require to re-train the adaptive artificial intelligence or machine learning arrangement. Since the new input is treated as the supplementary input, the likelihood values (i.e. conditional probabilities or Bayes factor) of each gene variant-phenotype relationship is updated to reduce uncertainty and increase certainty of the Bayesian mappings. This further enhances the accuracy of the screening system so that the results can be conveniently used by the physician for various applications.

Alternatively, optionally, the screening system further generates clinical report summary that provides actionable assessment for the subject. The clinical report summary summarises or gives an account of analysis of the compiled genome of the subject to confirm either presence or absence of a medical condition (i.e. a phenotype caused due to one or more gene variants as indicated in Bayesian mappings) with certain level of certainty so that appropriate remediation action may be taken. In other words, the clinical report summary is indicative of a confirmation or a denial of an existence of the medical condition of the subject when a probability is greater than a specified threshold to reduce uncertainty. Beneficially, the disclosed screening system outputs clinical report summary that enables to act on the assessed medical condition of the subject with increased certainty. For example, the medical condition of the subject is confirmed or denied with increased certainty. Thus, the clinical report summary generated by the screening system can be also employed in primary care and/or secondary care to treat the medical condition of the subject.

For example, the clinical report summary includes patient name, date of birth, Lab ID, phenotype summary, Year of birth (used in case of unborn child), family, clinical presentation, comments, data type, HPO terms, primary findings for decision support, secondary findings for decision support, and the like. The decision support information for phenotype summary provides determined phenotypic details, for example, “Micrognathia, Fetal akinesia, Non-immune hydrops fetalis, polyhydramnios”. The year of birth, for example, include “20-week scan”, i.e. in case of fetus. The clinical presentation, for example, include “Fetal anomaly scan at 20 weeks detected for hydrops with polyhydramnios and contractures affecting all four limbs and absent foetal movement. Male foetus was stillborn at 26 weeks and autopsy revealed micrognathia, joint contractures and multiple pterygia”. The comments, for example, include “karyotype and chromosome microarray were normal”. The data type, for example, include exome sequencing. The HPO terms, for example, include “HP 0000347 ‘micrognathia’, HP 0001561 ‘polyhydramnios’, HP 0001989 ‘fetal akinesia sequence’, HP 0001790 ‘nonimmune hydrops fetalis’, HP 0002803 ‘congenital contracture. These provide enhanced decision support for assessment by a user, and also useful in primary and secondary care to avoid unnecessary tests, and costs associated with such additional tests, which may have been prescribed otherwise.

Moreover, the sequence of events that causes the output of the clinical report summary for the subject is traceable. This enables a health care professional to characterize and audit the output of the clinical report summary, which in turn increases the confidence of the health care professional to use the outputted diagnostic information for deciding a next course of medical action, which may have practical life-saving implications for the subject.

Optionally, the control circuitry is further configured, namely is further operable, to generate a recommendation based on the clinical report summary to remediate the medical condition of the subject. Optionally, a treatment plan may be recommended based on the clinical report summary. Optionally, the generated recommendation and the decision-making process for the clinical report summary is communicated to one or more preconfigured external electronic devices (e.g. registered smartphones of a physician) for provisioning of personalized remediation in a primary care or a secondary care to the subject. It will be appreciated that the “one or more preconfigured external electronic devices” refer to, for example, a user equipment. Additionally, optionally, the one or more preconfigured external electronic devices are associated with providers of the primary care or providers of the secondary care, or both. It will be appreciated that the providers of the primary care include, for example, independently-practicing doctors, and the providers of the secondary care include, for example, district hospitals, community health centres (centers), and the like.

Optionally, the control circuitry is further configured, namely is further operable, to output an alert when the decision support information or the clinical report summary outputted by the screening system have the probability less than the specified threshold. Specifically, alerting prevents the user of the screening system to take substantial decisions based the outputted decision support information (or the clinical report summary). Moreover, the alert may further provide a reminder of their being insufficient information in the multi-dimensional data structure.

According to an embodiment, the screening system, when in operation, adds a copy of the one or more gene variants and phenotype information of the subject to augment the historical data samples of other subjects including their corresponding phenotype information of the other subjects and their one or more gene variants. Based on the currently executed gene variant interpretation that finds the one or more phenotype-gene variant relationships, such findings are useful for future gene variant interpretation for another subject, such as a new patient. Thus, the copy of the one or more gene variants and phenotype information of the subject is added in a database of the historical data samples of other subjects including their corresponding phenotype information of the other subjects and their one or more gene variants. Such copy of the one or more gene variants and phenotype information of the subject are added as further learnings in the screening system, thus the screening system becomes more robust over time. Alternatively stated, the corpus of data of new individuals grows with time and aggregation reduces uncertainty and increases accuracy in subsequent gene variant interpretation for new subjects.

According to an embodiment, the screening system is configured, namely operable, to process the historical data samples of other subjects including their corresponding phenotype information of the other subjects and their one or more gene variants to enable the historic data samples to be communicated and shared with other screening systems, to allow for data to be shared to increase a total size of the historical data samples of other subjects. The aforesaid screening system and the aforesaid method provides a mechanism that enables communication of the historic data samples (i.e. sensitive medical data) with other screening systems without compromising security and confidentiality of the other subjects. The screening system at a first location potentially transmits/receives such historic data samples from one or more other screening system situated at same or one or more other locations. Moreover, the historical data samples are shared with other screening systems by way of a data communication network. It will be appreciated that the data communication network may be wired or wireless, or a combination of both. Examples of the data communication networks include, but are not limited to, local area networks (LANs), radio access networks (RANs), metropolitan area networks (MANS), wide area networks (WANs), all or a portion of a public network such as the global computer network known as the Internet®, a private network, a cellular network and any other communication system or systems at one or more locations.

According to an embodiment, the screening system, when in operation, obfuscates the historical data samples of other subjects so that an identity of the other subjects is not discernible, wherein obfuscation is performed using at least one of: data extrapolation to generate additional synthetic subject data, or data blurring. In an example, the screening system obfuscates (i.e. obscures) datapoints of the multi-dimensional data structure before sharing with another screening system in the obscured form. Beneficially, obscuring the datapoints allows for exchange of characteristics relating to information associated with different subjects without explicit exchange of the sensitive information or specific person identifiable information. Therefore, the prevention of explicit exchange of information prevents security risks associated with such critical data and further exchange of characteristics relating to the information associated with the different subjects substantially reduces time and effort required for learning of the other screening system(s) which receives such information related to historical data samples. Moreover, such exchange of characteristics relating to the historical data samples reduces uncertainty in gene variant interpretation at the other screening system(s) which receives such information related to historical data samples, and also makes the process of generation of Bayesian mappings defining one or more gene variants-phenotype relationships for a new subject less time-intensive, which is useful and has life-saving implications in case of critical health conditions of the new subject. Moreover, the exchange of the historical data samples of other subjects in an obscured form reduces a computing power required for the process of finding new gene variants-phenotype relationships for a new subject at the other screening system(s) which receives such information, since it is not required to be trained again from start.

Optionally, the control circuitry is configured, namely is operable, to apply data extrapolation to generate additional synthetic subject data in order to obfuscate the historical data samples of other subjects so that an identity of the other subjects is not discernible. Generally, data extrapolation refers to estimation of a new value based on extending a known sequence of values or known facts. In other words, data extrapolate enables to infer additional synthetic subject data that is not explicitly stated from existing information of historical data samples. In this regard, in an example, instead of storing actual gene variant-phonotypic relationships of each subject of the different subjects as is in a database server of the screening system, the historical data samples are potentially stored as additional synthetic subject datapoints (not understandable by human to identify a subject) in the multi-dimensional data structure. The additional synthetic subject datapoints, even if identified by back tracing during audit, cannot be used to ascertain the identify the subject in any manner.

Alternatively, optionally, interpolation of data points in historical data samples may be used to derive new insights. For example, it is analyzed that a gene variant ‘A’ of original gene ‘X’ at a first gene locus is responsible for disease ‘B’ and a gene variant ‘B’ of the original gene ‘X’ also causes the same disease ‘B’. Further, it is found that a certain example stretch of a gene, for example ‘AAAAATAAAAAT’ (note: this is a fictitious example, and does not represent actual read DNA sequence information), when present as variants at any coding regions of the gene makes the gene potentially pathogenetic (in other words the repeat elements ‘AAAAAT’ are actual causes of manifest of disease in a human subject. Thus, if any other near variations of the gene ‘X’ (i.e. other than gene variants ‘A’ and ‘B’), having same stretch of gene (e.g. AAAAATAAAAAT), it can be readily associated with the disease ‘B’ for any new subjects. In another example, instead of actual data point that defines a quantitative information of a given subject, a range of the quantitative information or a near value of the datapoint is potentially used as a result of interpolation. Typically, locations of such gene variants in a genome provides an indication if those gene variants are more likely to manifest a phenotype or not. Furthermore, at a certain point in life, some genes are not expressed, while some specific genes are expressed in higher quantities (i.e. gene expressions levels are more at certain points of time, or due to external environment factors, or change in food or sleeping habits). Thus, such data points associated with other data points potentially provide a good understanding of how likely a given gene variant being interpreted will manifest into a phenotype in future with increase in age of the subject (i.e. a disease or manifest into a system of disease).

Optionally, the control circuitry is configured, namely is operable, to apply data blurring in order to obfuscate the historical data samples of other subjects so that an identity of the other subjects is not discernible. The historical data samples of other subjects are masked such that person identifiable data is obfuscated. Examples of a person identifiable data include, but are not limited to: name, location, patient ID, age, gender, disease suffering from, an actual genomic sequence of subjects, and the like. Optionally, the control circuitry hashes the data of historical data samples, using hash functions, which is a one-way operation, which prevents to “reverse engineer” the original data by simply analyzing the hashed values. Beneficially, obscuring the data of historical data samples allows for exchange of critical medical data associated with the different subjects without hampering security of the critical data and further by following several standardized norms of data transfer, data protection, and confidentiality.

Optionally, the other screening systems that receives the obfuscated historical data samples of other subjects cannot unscramble information such as identity, current status of any of the subjects, and the like. However, the obfuscated historical data samples of other subjects allow the other screening systems to update corresponding multi-dimensional data structure present therein, to quickly learn, for example, identification of gene variant-phenotype associations, and so forth.

Optionally, the control circuitry is further configured to communicate control instructions that comprises a set of machine-readable parameters along with the obfuscated historical data samples of other subjects to the other screening systems. In this regard, the screening system communicates the control instructions for enabling learning of corresponding artificial intelligence (AI) or machine learning (ML) arrangement in the other screening systems using the received set of machine-readable parameters. In an example implementation, the control instructions comprising machine-readable parameters are machine-learning algorithms, wherein the machine learning algorithms include weights associated with each layer of operation thereof. In another example implementation, the control instructions comprising machine-readable parameters are decryption keys for unscrambling of information from the obscured datapoints, wherein the unscrambled information is used by the other screening systems.

Optionally, a computing arrangement operated by each of the other screening systems re-calibrates Bayesian mappings based on a combination of the control instructions that comprises the set of machine readable-parameters, and the obfuscated historical data samples of other subjects, wherein the re-calibration reduces the stochastic errors and stochastic distortion and increases certainty in gene variant interpretation for new subject.

According to an embodiment, the screening system includes a functionality for user-selection of a subset of the historical data samples of other subjects to test for a sensitivity or convergence of the one or more phenotype-gene variant relationships to specific historical data samples. The screening system allows to select a subset or adjust the historical data samples of other subjects instead of using the default set of historical data samples of other subjects. In an implementation, such selection is executed automatically based on a match in gender, input biological sample from which genetic material isolated, age of subject, and the like, between the complied genomic sequence representative of the subject and each of the other historical data samples of other subjects. In another implementation, the graphical user interface is used to select and deselect (i.e. opt in or opt out) certain historical data samples in the set of samples of the multi-dimensional data structure. The opt in or opt out of certain historical data samples is based on the sensitivity of the one or more phenotype-gene variant relationships to specific historical data samples. For example, if selecting one historical sample drastically increases or reduces the number and probability of one or more phenotype-gene variant relationships, such a historical data sample is potentially re-evaluated for presence of any errors, and accordingly opted in or opted out, and thus and thus the risk of misinterpretation of gene variants for the subject is significantly reduced.

It will be appreciated that one or more gene variants can give rise to phenotypes that are any one of:

(i) benign;

(ii) likely benign;

(iii) unknown (VUS);

(iv) likely pathogenic; and

(v) pathogenic.

In practice, a variant is actually either pathogenic for a given phenotype or not. Thus, in effect, the middle three categories (ii) to (iv) are “errors” in that they do not represent reality, but only degrees of uncertainty. Thus, the model employed is capable of also reducing an occurrence of such “errors”.

According to an embodiment, the screening system, when in operation, determines a convergence of the one or more phenotype-gene variant relationships as a function of selection of the subset to determine an asymptotic trend of convergence in generation of the one or more phenotype-gene variant relationships. A threshold limit is potentially set, namely defined or adjusted, when selection of the subset is performed, and during the selection and deselection, the asymptotic trend of convergence is determined in generation of the one or more phenotype-gene variant relationships. It is observed if the change in one or more phenotype-gene variant relationships determined is an abrupt change or not based on the asymptotic trend. That is, asymptotic trend accounts for the abrupt change that may adversely influence gene variant interpretation results. The asymptotic trend of convergence, in effect, corresponds to an incremental reduction of uncertainty in gene variant interpretation to find one or more phenotype-gene variant relationships. In turn, the accuracy for decision support and provides improved assistance to a user, for example, to reduce the uncertainty of diagnosis of a medical condition or disease of the new subject may be improved.

In an exemplary implementation, the disclosed screening system uses the multi-dimensional data structure to effectively and efficiently reduce a sensitivity of the gene variant interpretations to the stochastic errors and stochastic distortion pre-existent in the input data and thus the risk of misinterpretation of gene variants for the subject is significantly reduced. Beneficially, the control circuitry determines sensitivity level of sparse datapoints in the multi-dimensional data structure, identifies a plurality of parameters (e.g. software faults or erroneous rules defined in software, and makes a selection of the subset of the historical data samples of other subjects to test for a sensitivity or convergence of the one or more phenotype-gene variant relationships to specific historical data samples) that causes abrupt changes and adversely influence gene variant interpretation results, and iteratively re-calibrates the plurality of parameters such that a sensitivity of the gene variant interpretation to the stochastic errors and distortions is reduced in each iteration. Thus, the disclosed screening system is improved to perform automatically gene variant interpretation with increased accuracy in each iteration as the sensitivity of the gene variant interpretation to the stochastic errors and distortions is reduced in each iteration. Furthermore, the re-execution of the gene variant interpretation provides improved gene variant-phenotypic relationships, which have further reduced sensitivity of the gene variant interpretation to the stochastic errors and stochastic distortion (i.e. almost nullifies the adverse effect of stochastic errors and stochastic distortion). The aforesaid screening system and the aforesaid screening method thus provided improved gene variant-phenotypic relationships, which are intermediate results for providing assistance to a clinical expert or act as a decision support tool for a clinical expert for many practical applications. Moreover, the screening system enables iterative re-calibration of the plurality of parameters (e.g. total number of historical data samples selected) that causes abrupt changes and adversely influence gene variant interpretation results, to iteratively correct the identified system faults of the screening system, which in turn increases the accuracy for decision support and provides an improved assistance to a user, for example, to reduce uncertainty of diagnosis of a medical condition or disease of new subject.

In an example, the term “sparse datapoints” refers to thinly dispersed datapoints in the multi-dimensional data structure, in which certain expected values in a dataset are missing or less. Sparse datapoints are created due to a plurality of parameters that may include, but are not limited to diverse sources and formats of data from which the multi-dimensional data structure, is generated. Approximately 99.96% of the multi-dimensional data structure may be sparse or without any datapoints. This may be due at least to the size of the variant pool and the limited availability of datapoints associated with each variant. Sparse datapoints usually result in higher sensitivity level to a particular input datapoint than other datapoints when fed to the screening system. For example, the number of historical data samples selected are not statistically relevant. The sensitivity level is potentially defined as a lower level, a medium level or a higher level of sensitivity depending upon the changes in a generated result due to a particular input. For example, results generated by the Bayesian mappings potentially exhibit a higher sensitivity level to particular input datapoint (e.g. a certain measured value or one of historical data sample in the set of data samples) for a patient than other datapoints, which potentially result in a sudden spike or fall in the output of the screening system (e.g. change in one or more phenotype-gene variant relationships due to changes in specific historical data samples). Such datapoints and the associated sensitivity to such datapoints are identified. Thus, the sensitivity level of a datapoint is indicative of potential faults in the screening system. The sensitivity analysis is typically computationally intensive.

According to an embodiment, in order to achieve computational efficiency, the plurality of datapoints including annotations stored in the multi-dimensional data structure, are first categorized in data type and the time of receipt of information. For example, all datapoints of phenotype information observed from abnormality scans from a particular medical equipment, are assigned same category. Thus, while testing sensitivity for one datapoint, if the output result, for example, generated confidence score changes drastically when just one datapoint is changed, then all datapoints of one category, such as the datapoints or annotations obtained from abnormality scans are considered highly sensitive and subject to further analysis for second stage. The assignment of the same data type to a group of datapoints originating from a same data source, a same type of file format, significantly reduces the computational load of the screening system. In an instance, when high sensitivity is found, further tests are performed to find whether the high sensitivity is due to a data error or a system fault of the screening system. The system fault is potentially a programming fault, a data-structure fault, or a fault in defining rules of the first artificial intelligence-based system, the second artificial intelligence-based system, or the Bayesian mapping arrangement, or both.

Optionally, the control circuitry is further configured, namely is further operable, to identify a plurality of parameters that causes abrupt changes and adversely influence gene variant interpretation results by the Bayesian mappings. The plurality of parameters corresponds to system settings parameters and a plurality of defined rules that are used process the received input, and to finally generate the gene variant interpretation which includes the one or more gene variant-phenotypic relationships. If there is a difference in the output generated from the expected output, then the plurality of parameters that are responsible for such spurious input/output behaviour of the screening system is determined. The term “abrupt changes” refers to a percentage change that is above a specified threshold in a system output from the screening system when a particular datapoint in the first multi-dimensional structure is fed as input to the system. For example, a confidence score generated by the screening system in the first iteration is ‘X’ percent, and the threshold may be set as 10%. If a new datapoint is fed in the first multi-dimensional structure, which increases or decreases the current confidence score that describe, for example, probability of a phenotype-gene variant relationship, by 10% or more than 10% (set threshold), then such change due to the datapoint input is said to be an abrupt change. However, If the new datapoint is fed in the first multi-dimensional structure, which increases or decreases the current confidence score by less than 10%, then such change due to the datapoint input is said to be a non-abrupt change. It is to be appreciated that instead of 10%, any percentage in a range of 1% to 100%, may be set as threshold depending on user-preference, and after a few experimentations (e.g. using the difference in the output generated from the expected output), an appropriate threshold level is potentially defined. Thus, all the parameters including selection of a subset of historical data samples that causes abrupt changes and adversely influence gene variant interpretation results on input of the datapoints (data elements) in various dimensions of the multi-dimensional data structure, are identified for further use.

Optionally, the control circuitry is further configured, an iterative manner, to re-calibrate the plurality of parameters that causes abrupt changes and adversely influence the gene variant interpretation results such that a sensitivity of the gene variant interpretation to the stochastic errors and distortions is reduced in each iteration. Once the plurality of parameters that causes abrupt changes and adversely influence the gene variant interpretation results are identified, an adjustment of the identified parameters is performed. In order to re-calibrate the plurality of parameters, a sequence of events starting from input of a datapoint to all subsequent events of processing the datapoint in each layer or processing stage is checked, until the final output. The event to event tracking in the sequence of events provides a detailed understanding of the parameters that are potentially not calibrated optimally for such type of datapoint. When the difference in the output generated from the expected output is minimal, or almost zero, it is considered that the re-calibration of the plurality of parameters is achieved, and the sensitivity of the gene variant interpretation to the stochastic errors and distortions is reduced or almost nullified.

Optionally, the control circuitry is further configured, namely is further operable, to re-execute the gene variant interpretation for the subject having re-calibrated plurality of parameters, wherein the gene variant interpretation includes updated gene variant-phenotypic relationships, wherein the updated gene variant-phenotypic relationships have reduced sensitivity of the gene variant interpretation to the stochastic errors and distortions. If any erroneous datapoint is found associated with the identified plurality of parameters, that datapoint is potentially flagged and ignored in a next iteration of the re-calibration of the plurality of parameters. Alternatively, if the parameter that abruptly changes the output of the screening system is a rule that define gene variant-phenotypic relationships, then the calibration of the rule automatically removes the erroneous datapoints, and the multi-dimensional data structure is updated in a next iteration (e.g. the second iteration). Optionally, the Bayesian mappings rules and underlying plurality of probabilities of an occurrence of a relation between a gene variant and a phenotype based on prior knowledge of conditions that is potentially related to the gene variant-phenotype relation is adjusted until the difference between the expected output (ground truth) and generated output is minimum or zero. The identification and iterative re-calibration of a plurality of parameters that causes abrupt changes and adversely influence gene variant interpretation results automatically self-corrects the system faults related to spurious input/output behaviour, which in turn further improves the accuracy of the screening system and makes it ready to perform analysis of genome information (genome or exome) for a new subject. If over-sensitivity is found during alignment of the plurality of genomic sequences representative of individual's DNA to a reference genome (e.g. mismatch greater than a specified percentage), then in some cases, re-sequencing of the given individual's DNA is potentially required, and accordingly alert is generated.

The present disclosure also relates to the method as described above. Various embodiments and variants disclosed above apply mutatis mutandis to the method.

According to an embodiment, the method is characterized in that the method further includes using the screening system to generate a graphical representation of the one or more phenotype-gene variant relationships for user-editing and adjustment on a graphical user interface.

According to an embodiment, the method is characterized in that the method further using the screening system to generate one or more Bayesian mappings describing one or more phenotype-gene variant relationships that have a probability that exceeds one or more threshold criteria.

According to an embodiment, the method is characterized in that the method further includes employing an adaptive artificial intelligence or machine learning arrangement to assist the screening system to generate the one or more Bayesian mappings.

According to an embodiment, the method is characterized in that the method further includes using the control circuitry to associate the one or more generated Bayesian mappings describing one or more phenotype-gene variant relationships with a secondary database of historical medical reports to identify one or more historical medical reports that are related in subject matter to the one or more generated Bayesian mappings, and to present the identified one or more historical medical reports as a graphical list on the graphical user interface. The medical reports beneficially include past gene variant classifications, for example.

According to an embodiment, the method is characterized in that the method further includes arranging for the screening system, when in operation, to use the identified one or more generated Bayesian mappings and the identified one or more historical medical reports to provide decision support information in respect of the subject.

According to an embodiment, the method is characterized in that the method further includes arranging for the screening system to process, when in operation, the one or more gene variants present in the compiled genome representative of the subject relative to the reference genome based to reduce stochastic errors due to at least one of: indels, call number variations (CNV's), substantial palindromes, incorrectly identified or mis-classified phenotypes.

According to an embodiment, the method is characterized in that the method further includes arranging for the screening system, when in operation, to add a copy of the one or more gene variants and phenotype information of the subject to augment the historical data samples of other subjects including their corresponding phenotype information of the other subjects and their one or more gene variants.

According to an embodiment, the method is characterized in that the method further includes arranging for the screening system to process the historical data samples of other subjects including their corresponding phenotype information of the other subjects and their one or more gene variants to enable the historic data samples to be communicated and shared with other screening systems, to allow for data to be shared to increase an total size of the historical data samples of other subjects.

According to an embodiment, the method is characterized in that the method further includes arranging for the screening system, when in operation, to obfuscate the historical samples of other subjects so that an identity of the other subjects is not discernible, wherein obfuscation is performed using at least one of: data extrapolation to generate additional synthetic subject data, data blurring.

According to an embodiment, the method is characterized in that the method further includes arranging for the screening system to include a functionality for user-selection of a subset of the historical data samples of other subjects to test for a sensitivity or convergence of the one or more phenotype-gene variant relationships to specific historical data samples.

According to an embodiment, the method is characterized in that the method further includes arranging for the screening system, when in operation, to determine a convergence of the one or more phenotype-gene variant relationships as a function of selection of the subset to determine an asymptotic trend of convergence in generation of the one or more phenotype-gene variant relationships.

### DETAILED DESCRIPTION OF THE DRAWINGS

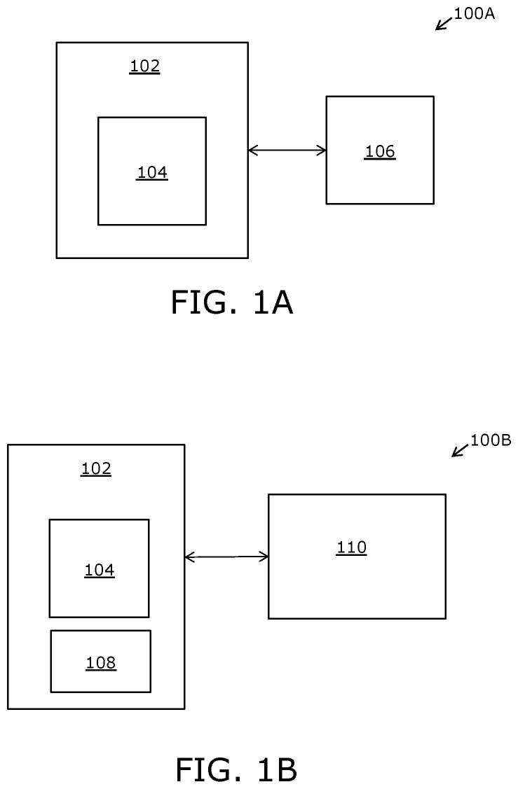

Referring to FIG. 1A, there is shown a block diagram that illustrates a network environment 100A of a screening system 102, in accordance with an embodiment of the present disclosure. The screening system 102 comprises a control circuitry 104. A sequencing apparatus 106 is communicatively coupled to the screening system 102. The control circuitry 104, when in operation, receives a plurality of genomic sequences of a plurality of genomic fragments of at least one biological sample from a subject that has been sequenced in the sequencing apparatus 106. The plurality of genomic sequences potentially includes stochastic errors and stochastic distortion. The control circuitry 104, when in operation, further aligns the plurality of genomic sequences to a reference genome to generate from the aligned genomic sequences a compiled genome representative of the subject. The control circuitry 104 is further configured, namely is further operable, to determine one or more gene variants present in the compiled genome representative of the subject relative to the reference genome based on a difference between the reference genome and the compiled genome representative of the subject. The control circuitry 104 is further configured, namely operable, to acquire phenotype information from an observation of the subject; the observation is performed, for example, by a medical practitioner or nurse. The phenotype information is potentially in the form of phenotypic codes that indicates a disorder.

The control circuitry 104, when in operation, generates a multi-dimensional data structure that includes the one or more gene variants in respect of a first dimension; the phenotype information in respect of a second dimension; and a set of data samples in respect of a third dimension, wherein the set of data samples includes the compiled genome sequence representative of the subject, and corresponding historical data samples of other subjects including their corresponding phenotype information of the other subjects and their one or more gene variants. The control circuitry 104 is configured, namely is operable, to execute a gene variant interpretation using a correlation function to find one or more phenotype-gene variant relationships based on the generated multi-dimensional data structure. The use of the multi-dimensional data structure reduces a sensitivity of the gene variant interpretation to the stochastic errors and stochastic distortion.

It may be understood by a person skilled in the art that FIG. 1A includes a simplified illustration of the screening system 102 for sake of clarity only, which should not unduly limit the scope of the claims herein. The person skilled in the art will recognize many variations, alternatives, and modifications of embodiments of the present disclosure.

Referring next to FIG. 1B, there is shown a block diagram that illustrates a network environment 100B that includes multiple screening systems, in accordance with another embodiment of the present disclosure. FIG. 1B is described in conjunction with elements from FIG. 1A. The network environment 100B includes the screening system 102 and another screening system 110. There is further shown the control circuitry 104 and a machine learning arrangement 108 in the screening system 102. The screening system 102 employs the machine learning (ML) arrangement 108 to generate the one or more Bayesian mappings that describe one or more phenotype-gene variant relationships.

In accordance with an embodiment, the control circuitry 104 of the screening system 102 is configure, namely is operable, to process historical data samples of other subjects that includes corresponding phenotype information of the other subjects and their one or more gene variants. The historical data samples of other subjects form a part of the multi-dimensional data structure stored in the screening system 102. The historical data samples of other subjects are processed to obfuscate the historical data samples so that an identity of the other subjects is not discernible. Thereafter, the obfuscated historic data samples are communicated (i.e. shared) with other screening systems, such as the screening system 110, to allow for data to be shared to increase a total size of the historical data sample of other subjects that is used in the gene variant interpretation.

It may be understood by a person skilled in the art that FIG. 1B includes a simplified illustration of the screening systems 102 and 110 for sake of clarity, which should not unduly limit the scope of the claims herein. The person skilled in the art will recognize many variations, alternatives, and modifications of embodiments of the present disclosure.

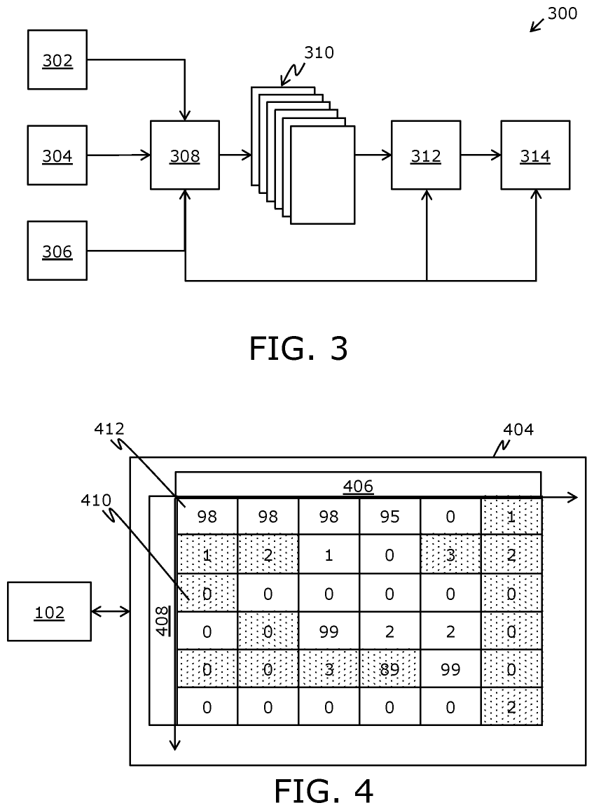

Referring to FIG. 3 there is shown a schematic illustration of a screening system 300, in accordance with an exemplary embodiment of the present disclosure. As shown, the screening system 300 comprises a control circuitry 308. The control circuitry 308, when in operation, generates a multi-dimensional data structure 310. The multi-dimensional data structure 310 is generated based on the one or more gene variants 302 of a subject determined by the control circuitry 308, acquired phenotype information 304 that is derived from observation of the subject, and a set of data samples 306. The multi-dimensional data structure 310 includes the one or more gene variants 302 in respect of a first dimension, the phenotype information 304 in respect of a second dimension; and the set of data samples in respect of a third dimension. The set of data samples includes a compiled genome sequence representative of the subject, and historical data samples of other subjects including their corresponding phenotype information of the other subjects and their one or more gene variants.

The control circuitry 308 is further configured, namely is further operable, to execute a gene variant interpretation 312 using a correlation function to identify, namely to find, one or more phenotype-gene variant relationships based on the generated multi-dimensional data structure 310. In some embodiments, the control circuitry 308 is further configured, namely is further operable, to output a confidence score 314 that indicates at least a causative element of an observed medical condition of the subject represented by a phenotype (in one or more phenotype-gene variant relationships) to be a particular gene variant (or two or more gene variants) which is unable to encode a functional protein resulting in the phenotype. The confidence score 314 indicates the particular gene variant (or the two or more gene variants) to be a confirmed cause of the phenotype in question when the confidence score is greater than a specified threshold.

Referring next to FIG. 4, there is shown a schematic illustration of an exemplary matrix 404 depicting phenotype-variant relationship probabilistically, associated with a screening system 102, in accordance with an embodiment of the present disclosure. As shown, the matrix 404 depicts a list of gene variants 406 in a first axis (i.e. in respect of a first dimension) and a list of phenotypes 408 in a second axis (i.e. in respect of a second dimension). Furthermore, the matrix 404 is populated with numeric values 410 and 412. The screening system 102, when in operation, executes a gene variant interpretation using a correlation function to find one or more phenotype-gene variant relationships. The set of data samples are also used in gene variant interpretation (not shown). In the gene variant interpretation, the matrix 404 generates the numeric values 410 and 412 to define a probability and quantify a level of certainty around it (i.e. quantify the likelihood of a gene variant responsible for a phenotype). Moreover, the numeric values 410 and 412 refer to a probability of pathogenicity, where a value close to ‘0’ indicates zero probability and a value close to ‘100’ indicate very high probability (e.g. value greater than 90 may indicate a confirmation). Such upgradation of the numeric values 410 and 412 close to ‘0’ or ‘100’ enables reduction of uncertainty in finding a phenotype-gene variant relationship of a subject.

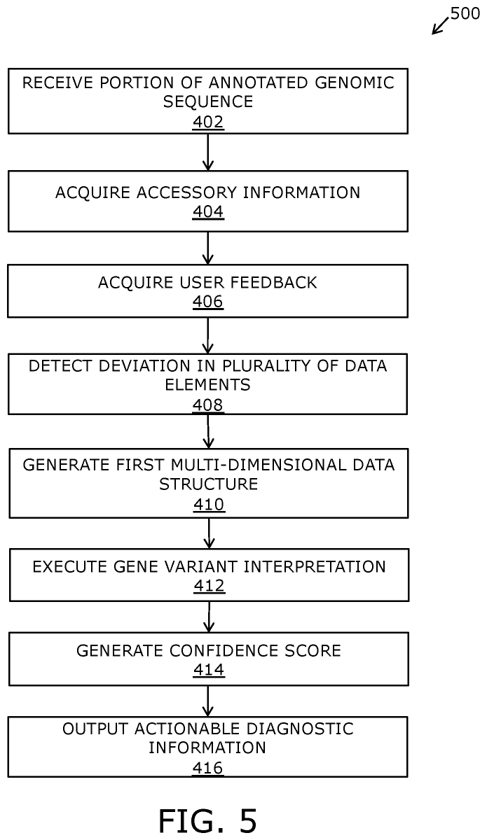

Referring next to FIG. 5, there is shown an illustration of a flowchart 500 depicting steps of a screening method, in accordance with an embodiment of the present disclosure. The method is depicted as a collection of steps in a logical flow diagram, which represents a sequence of steps that can be implemented in hardware, software, or a combination thereof, for example as aforementioned. The method is implemented in a screening system that comprises control circuitry.

At a step 502, a control circuitry is used to receive a plurality of genomic sequences of a plurality of genomic fragments of at least one biological sample from a subject that has been sequenced in a sequencing apparatus, for example an Illumina® or Qiagen® proprietary sequencer, wherein the plurality of genomic sequences includes stochastic errors and stochastic distortion. At a step 504, the plurality of genomic sequences is aligned to a reference genome to generate from the aligned genomic sequences a compiled genome representative of the subject. At a step 506, one or more gene variants present in the compiled genome representative of the subject are determined relative to the reference genome based on a difference between the reference genome and the compiled genome representative of the subject. At a step 508, phenotype information is acquired from an observation of the subject. At a step 510, a multi-dimensional data structure is generated that includes:

(a) the one or more gene variants in respect of a first dimension,

(b) the phenotype information in respect of a second dimension, and

(c) a set of data samples in respect of a third dimension, wherein the set of data samples includes one or more gene variants determined from the compiled genome sequence representative of the subject, and corresponding historical data samples of other subjects including their corresponding phenotype information of the other subjects and their one or more gene variants.

At a step 512, a gene variant interpretation is executed using a correlation function to identify, namely to find, one or more phenotype-gene variant relationships based on the generated multi-dimensional data structure, wherein using the multi-dimensional data structure reduces a susceptibility of the gene variant interpretation to be affected by the stochastic errors and stochastic distortion.

The steps 502 to 512 are only illustrative and other alternatives can also be provided where one or more steps are added, one or more steps are removed, or one or more steps are provided in a different sequence without departing from the scope of the claims herein.

In the foregoing, it will be appreciated the data sample for the subject, namely “patient data”, is made anonymous by converting using encryption some data fields to numbers and storing corresponding encryption keys securely. Moreover, it will be appreciated that the multi-dimensional data structure (model) that is generated, includes of a statistical measure of pathogenicity level (classification), using Bayesian inference (i.e. taking some classification information as previous known and then inferring the probability of a class for newly presented variants). The multi-dimensional data structure provides a model that reduces erroneous variant definitions (particularly the aforementioned ‘VUS’ classification, when in fact the variant will be either benign or pathogenic).

It is advantageous that the multi-dimensional data structure (namely model) is continuously updated with new patient information and new scientific information, thereby reducing an uncertainty and potential errors when identifying gene variant classifications. In embodiments of the present disclosure, genetic variants are identified where the pathogenicity classification given by the model has changed from a previous human-defined classification (namely error removed); there are beneficially flagged up past unsolved cases that are affected by such change (wherein such flagging up is likely to pertain to subjects having a classification as ‘Variants of Unknown Significance (VUS), to a prediction of benign or pathogenic).

Beneficially, the model enables identification of patient profiles that are most likely to have their variant classification error reduced (namely, least likely to be classified as VUS), for example patients that are experiencing a certain phenotype are male, etc. and are x % likely to be classifiable. Beneficially, embodiments of the present disclosure combine predictions from multiple models created with a similar structure, but using a different data source to further reduce the error or uncertainty.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A screening system comprising
a control circuitry that, when in operation:
receives a plurality of genomic sequences of a plurality of genomic fragments of at least one biological sample from a subject that has been sequenced in a sequencing apparatus, wherein the plurality of genomic sequences includes stochastic errors and stochastic distortion;
aligns the plurality of genomic sequences to a reference genome to generate from the aligned genomic sequences a compiled genome representative of the subject;
determines one or more gene variants present in the compiled genome representative of the subject relative to the reference genome based on a difference between the reference genome and the compiled genome representative of the subject,
acquires phenotype information from an observation of the subject,
wherein the control circuitry further:
generates a multi-dimensional data structure that includes:
the one or more gene variants in respect of a first dimension;
the phenotype information in respect of a second dimension; and
a set of data samples in respect of a third dimension, wherein the set of data samples includes one or more gene variants representative of the subject and their corresponding phenotype information, and corresponding historical data samples of other subjects including their one or more gene variants and their corresponding biological (for example, transcript) information;
executes a gene variant interpretation using a correlation function to identify one or more phenotype-gene variant relationships based on the generated multi-dimensional data structure, wherein using the multi-dimensional data structure reduces a susceptibility of the gene variant interpretation to be affected by the stochastic errors and stochastic distortion.

2. The screening system of claim 1, characterized in that the screening system is operable to generate a graphical representation of the one or more phenotype-gene variant relationships for user-editing and adjustment on a graphical user interface, wherein the graphical representation also provides a visual indication of strengths of correlation.

3. The screening system of claim 1, wherein the screening system generates one or more Bayesian mappings describing one or more phenotype-gene variant relationships that have a probability that exceeds one or more threshold criteria.

4. The screening system of claim 3, wherein the screening system employs an adaptive artificial intelligence or machine learning arrangement to generate the one or more Bayesian mappings.

5. The screening system of claim 2, wherein the screening system generates one or more Bayesian mappings describing one or more phenotype-gene variant relationships that have a probability that exceeds one or more threshold criteria; and
wherein the control circuitry is operable to associate the one or more generated Bayesian mappings describing one or more phenotype-gene variant relationships with a secondary database of historical medical reports to identify one or more historical medical reports that are related in subject matter to the one or more generated Bayesian mappings, and to present the identified one or more historical medical reports as a graphical list on the graphical user interface.

6. The screening system of claim 5, wherein the screening system, when in operation, uses the identified one or more generated Bayesian mappings and the identified one or more historical medical reports to provide decision support information in respect of the subject.

7. The screening system of claim 1, wherein the screening system processes, when in operation, the one or more gene variants present in the compiled genome representative of the subject relative to the reference genome based to reduce stochastic errors due to at least one of: indels, call number variations (CNV's), substantial palindromes, incorrectly identified or mis-classified phenotypes.

8. The screening system of claim 1, wherein the screening system, when in operation, adds a copy of the one or more gene variants and the phenotype information of the subject to augment the historical data samples of other subjects including their corresponding phenotype information of the other subjects and their one or more gene variants.

9. The screening system of claim 1, wherein that the screening system is operable to process the historical data samples of other subjects including their corresponding phenotype information of the other subjects and their one or more gene variants to enable the historic data samples to be communicated and shared with other screening systems, to allow for data to be shared to increase a total size of the historical data samples of other subjects.

10. The screening system of claim 9, wherein that the screening system, when in operation, obfuscates the historical data samples of other subjects so that an identity of the other subjects is not discernible, wherein obfuscation is performed using at least one of: data extrapolation to generate additional synthetic subject data, or data blurring.

11. The screening system of claim 1, wherein that the screening system includes a functionality for user-selection of a subset of the historical data samples of other subjects to test for a sensitivity or convergence of the one or more phenotype-gene variant relationships to specific historical data samples.

12. The screening system of claim 11, wherein that the screening system, when in operation, determines a convergence of the one or more phenotype-gene variant relationships as a function of selection of the subset to determine an asymptotic trend of convergence in generation of the one or more phenotype-gene variant relationships.

13. A method of operating a screening system, wherein the method comprises:
(i) using a control circuitry to receive a plurality of genomic sequences of a plurality of genomic fragments of at least one biological sample from a subject that has been sequenced in a sequencing apparatus, wherein the plurality of genomic sequences includes stochastic errors and stochastic distortion;
(ii) aligning the plurality of genomic sequences to a reference genome to generate from the aligned genomic sequences a compiled genome representative of the subject;
(iii) determining one or more gene variants present in the compiled genome representative of the subject relative to the reference genome based on a difference between the reference genome and the compiled genome representative of the subject;
(iv) acquiring phenotype information from an observation of the subject;
(v) generating a multi-dimensional data structure that includes:
the one or more gene variants in respect of a first dimension;
the phenotype information in respect of a second dimension; and
a set of data samples in respect of a third dimension, wherein the set of data samples includes the one or more gene variants representative of the subject their corresponding phenotype information, and corresponding historical data samples of other subjects including their one or more gene variants and their corresponding biological (for example transcript) information;
(vi) executing a gene variant interpretation using a correlation function to identify one or more phenotype-gene variant relationships based on the generated multi-dimensional data structure, wherein using the multi-dimensional data structure reduces a susceptibility of the gene variant interpretation to be affected by the stochastic errors and stochastic distortion.

14. The method of claim 13, wherein the method further includes using the screening system to generate a graphical representation of the one or more phenotype-gene variant relationships for user-editing and adjustment on a graphical user interface.

15. The method of claim 13, wherein the method includes using the screening system to generate one or more Bayesian mappings describing one or more phenotype-gene variant relationships that have a probability that exceeds one or more threshold criteria.

16. The method of claim 15, wherein the method includes employing an adaptive artificial intelligence or machine learning arrangement to assist the screening system to generate the one or more Bayesian mappings.

17. The method of claim 14, wherein the method includes using the screening system to generate one or more Bayesian mappings describing one or more phenotype-gene variant relationships that have a probability that exceeds one or more threshold criteria; and
wherein the method includes using the control circuitry to associate the one or more generated Bayesian mappings describing one or more phenotype-gene variant relationships with a secondary database of historical medical reports to identify one or more historical medical reports that are related in subject matter to the one or more generated Bayesian mappings, and to present the identified one or more historical medical reports as a graphical list on the graphical user interface.

18. The method of claim 17, wherein that the method includes arranging for the screening system, when in operation, to use the identified one or more generated Bayesian mappings and the identified one or more historical medical reports to provide decision support information in respect of the subject.

19. The method of claim 13, wherein the method includes arranging for the screening system, when in operation, to add a copy of the one or more gene variants and phenotype information of the subject to augment the historical data samples of other subjects including their corresponding phenotype information of the other subjects and their one or more gene variants.

20. The method of claim 13, wherein the method includes arranging for the screening system to process the historical data samples of other subjects including their corresponding phenotype information of the other subjects and their one or more gene variants to enable the historical data samples to be communicated and shared with other screening systems, to allow for data to be shared to increase an total size of the historical data sample of other subjects.

21. The method of claim 20, wherein the method includes arranging for the screening system, when in operation, to obfuscate the historical data samples of other subjects so that an identity of the other subjects is not discernible, wherein obfuscation is performed using at least one of: data extrapolation to generate additional synthetic subject data, data blurring.

22. The method of claim 13, wherein the method includes arranging for the screening system to include a functionality for user-selection of a subset of the historical data samples of other subjects to test for a sensitivity or convergence of the one or more phenotype-gene variant relationships to specific historical data samples.

23. The method of claim 22, wherein that the method includes arranging for the screening system, when in operation, to determine a convergence of the one or more phenotype-gene variant relationships as a function of selection of the subset to determine an asymptotic trend of convergence in generation of the one or more phenotype-gene variant relationships.

24. A computer program product comprising a non-transitory computer-readable storage medium having computer-readable instructions stored thereon, the computer-readable instructions being executable by a computerized device comprising processing hardware to execute a method as claimed in claim 13.

25. The system of claim 3, wherein the multi-dimensional data structure corresponds to one or more models configured to generate the one or more Bayesian mappings, wherein the multi-dimensional data structure serves as input the one or more models.

26. The system of claim 4, wherein the adaptive artificial intelligence or machine learning arrangement comprises one or models configured to receive new patient data and/or new scientific information in relation to the multi-dimensional data structure for generating the one or more Bayesian mappings.

27. The system of claim 26, wherein the one or more Bayesian mappings incrementally update based on the new patient data and/or new scientific information received.

28. The system of claim 6, wherein the decision support information is selected from a group comprising: patient name, date of birth, Lab ID, phenotype summary, Year of birth, family, clinical presentation, comments, data type, HPO terms, primary findings for decision support, and secondary findings for decision support.

29. The system of claim 6, wherein the decision support information associated with the one or more gene variant-phenotype relationships for generating the Bayesian mappings are employed to train the adaptive artificial intelligence or machine or machine learning arrangement to update the Bayesian mappings.

30. The system of claim 1, wherein the one or more gene variants are associated with the phenotype information that are any one of: benign, likely benign, unknown (VUS), likely pathogenic, and pathogenic.

31. The method of claim 15, wherein the multi-dimensional data structure corresponds to one or more models configured to generate the one or more Bayesian mappings, wherein the multi-dimensional data structure serves as input the one or more models.

32. The method of claim 16, wherein the adaptive artificial intelligence or machine learning arrangement comprises one or models configured to receive new patient data and/or new scientific information in relation to the multi-dimensional data structure for generating the one or more Bayesian mappings.

33. The method of claim 32, wherein the one or more Bayesian mappings incrementally update based on the new patient data and/or new scientific information received.

34. The method of claim 18, wherein the decision support information is selected from a group comprising: patient name, date of birth, Lab ID, phenotype summary, Year of birth, family, clinical presentation, comments, data type, HPO terms, primary findings for decision support, and secondary findings for decision support.

35. The method of claim 18, wherein the decision support information associated with the one or more gene variant-phenotype relationships for generating the Bayesian mappings are employed to train the adaptive artificial intelligence or machine or machine learning arrangement to update the Bayesian mappings.
